(19) United States
(12) Patent Application Publication
(10) Pub. No.: US 2026/0083810 A1
(43) Pub. Date: Mar. 26, 2026

(54) METHODS AND COMPOSITIONS FOR REDUCING VISCERAL FAT IN A MAMMAL USING NATURALLY OCCURING PLANTS AND SPICES

(71) Applicants: Shil Kothari, Fairfield, CA (US); Julius Enyoug Oben, Cameroon (CM)

(72) Inventors: Shil Kothari, Fairfield, CA (US); Julius Enyoug Oben, Cameroon (CM)

(21) Appl. No.: 18/898,601

(22) Filed: Sep. 26, 2024

(51) Int. Cl. A61K 36/87; A61P 3/04

(52) U.S. Cl. CPC A61K 36/87; A61P 3/04; A61K 2236/17; A61K 2236/33

(57) ABSTRACT

Methods and compositions using Dichrostachys glomerata or Cissus quadrangularis separately to provide a variety of health benefits, including but not limited to, increase GLP-1 levels and reduce or decrease visceral fat, food intake, blood lipids, total cholesterol and total glucose in a mammal. In one embodiment, a composition for reducing visceral fat in a mammal is provided where the composition comprises an effective amount of Cissus quadrangularis provided as an oral dosage unit in the form of a pill, capsule, liquid, lozenge or tablet.

### FIELD OF THE DISCLOSURE

The present disclosure relates to methods and compositions for reducing visceral fat in a mammal using naturally occurring plants and spices. In at least one embodiment, an effective amount of a composition containing Cissus quadrangularis or Dichrostachys glomerata is provided to a mammal to naturally reduce visceral fat in the mammal and provide a variety of health benefits to the mammal.

### BACKGROUND

Obesity and the need to lose weight to improve a variety of health conditions and factors affect over 2.3 billion people of all ages globally (WHO, 2021). The development and progression of obesity and the variety of related health conditions involve a complex pathogenesis, and several pharmaceutical drugs have been developed to target the various pathways that control and affect obesity and weight gain. In recent years, dipeptidyl peptidase-4 (DPP-4) inhibitors or gliptins, such as sitagliptin, saxagliptin, and vildagliptin, have been introduced and are considered viable short to medium term obesity management options.

Glucagon-like peptide-1 (hereafter, “GLP-1”) is a naturally occurring hormone produced in the intestines in response to food intake. It plays a crucial role in glucose regulation, appetite control, and gut health. In recent years, research has unveiled a broader spectrum of its potential, off label, health benefits, extending beyond its initial discovery as a diabetes treatment.

While GLP-1 undeniably plays a role in weight loss, it's crucial to understand that weight loss products that claim to increase GLP-1 levels in humans are misleading and increasing GLP-1 through external means, like taking a supplement, might not lead to weight loss. GLP-1 is naturally produced in the body in response to food intake. Taking a supplement that increases GLP-1 or exogenous administration of GLP-1 might disrupt the body's natural regulatory mechanisms and not actually result in an increase in bio-available GLP-1 in the human body.

Additionally, overexposure to GLP-1 could potentially desensitize the GLP-1 receptors, diminishing the hormone's effectiveness over time. Weight loss is a multifaceted process influenced by various factors, including diet, exercise, genetics, and metabolism.

Dipeptidyl peptidase-4 (DPP-4) is an enzyme that plays a crucial role in regulating glucose homeostasis. It inactivates incretin hormones, glucagon-like peptide-1 (GLP-1) and glucose-dependent insulinotropic polypeptide (GIP), which stimulates insulin secretion and suppresses glucagon release. By inhibiting DPP-4, these incretin hormones have a prolonged lifespan, leading to improved glucose control. DPP-4 inhibition is thought to improve glucose control, lower blood sugar levels in people with type 2 diabetes, reducing the risk of diabetic complications such as heart disease, kidney disease, and nerve damage and potentially prevent the development of type 2 diabetes in individuals that are at high risk of becoming type 2 diabetics. Additionally, inhibiting DPP-4 may promote weight loss or prevent weight gain by increasing feelings of fullness and reducing appetite and improve insulin sensitivity, leading to better glucose uptake by cells and reduced fat storage. Furthermore, inhibiting DPP-4 may reduce the risk of heart attack and stroke by lowering blood pressure and improving blood lipid profiles, promote vasodilatation and reducing inflammation in blood vessels, protecting beta cells in the pancreas, which produce insulin, improve bone mineral density and reduce the risk of certain types of cancer.

While DPP-4 inhibition is associated with weight management benefits, it's crucial to understand the underlying mechanisms. DPP-4 inhibitors primarily improve glucose control and insulin sensitivity. While these effects may have an effect on or contribute to weight loss, the primary target and effect of products that inhibit DPP-4 is glucose metabolism, not fat breakdown or long-term weight loss.

Weight loss is influenced by various factors, including energy intake, energy expenditure, hormonal balance, and genetic predisposition. Targeting a single enzyme like DPP-4 would likely have limited to no impact on overall weight loss. Additionally, inhibiting DPP-4 can lead to increased insulin secretion. While beneficial for glucose control, excessive insulin can promote fat storage, particularly in individuals with insulin resistance.

Many pharmaceuticals that act as DPP-4 inhibitors have various side effects such as nausea, diarrhea, and upper respiratory tract infections and are poorly tolerated. Additionally, they tend to be less effective with time, making pharmaceutical DPP-4 inhibitors ineffective as a long-term solution to obesity, weight loss or weight management.

Unfortunately, dipeptidyl peptidase-4 (DPP-4) inhibitors or gliptins (and related GLP-1 pharmaceuticals) cause a variety of side effects and potentially serious complications and have been shown to lose effectiveness over extended periods of time. Gliptins inhibit DPP-4, an enzyme known to deactivate the Glucagon-Like Peptide-1 (GLP-1) hormone, contributing to the development and progression of obesity and other metabolic diseases. GLP-1 is thought to be one of the more important incretin hormones secreted in the L-cells of the gut for the maintenance of blood sugar homeostasis. It exhibits other pleiotropic effects through its receptors in the liver, brain, and stomach to delay gastric emptying, reduce appetite, and induce significant weight loss (Bloemendaal et al., 2014). In healthy individuals, GLP-1 has a half-life of 2>minutes due to the activities of DPP-4 (Ahrén and Schmitz, 2004). Some studies have observed higher DPP-4 levels in obese individuals (Valerio et al., 2017), further reducing the incretin effects of GLP-1. Gliptins are primarily created and prescribed to manage type 2 diabetes. Use of gliptins (GLP-1 pharmaceuticals) for weight loss is off label and not how these pharmaceutical drugs are intended to be used. However, since their weight loss effects have been shown to be quite significant, these pharmaceutical drugs are commonly used for weight loss and to treat obesity, despite the variety of side effects and potentially serious complications that often comes with their use and the fact that they have been shown to lose effectiveness over extended periods of time.

It is well known that many pharmaceutical and synthetically produced drugs often cause various side effects and contraindications. For conditions such as obesity, patients often require unique management options due to sensitivity and the high likelihood of co-morbidities. For instance, obese individuals are more vulnerable to various conditions and diseases, including pancreatitis and pancreatic cancer (Yadav and Lowenfels, 2013), whereas gliptins have been shown to be associated with a high incidence of acute pancreatitis (Tkáč and Raz, 2017). Gliptins also present other side effects such as upper respiratory infections, headache, urinary tract infections, arthralgia, and in severe cases, Stevens-Johnson syndrome (Filipatos et al., 2014). Cost-wise, gliptins are relatively expensive (Filipova et al., 2015) and do not offer a safe or good long-term solution for those who want to lose weight and improve their health, long term. Currently, gliptins are FDA-approved for the management of diabetes, not for weight loss or managing obesity. Prescribing gliptins for obesity may lead to higher demand and prices for these drugs, as well as scarcity. Hence, there is a significant need for a wider range of safe, cost-effective alternatives to gliptins that does not have serious side effects, does not lose its effectiveness over longer periods of time and provide a long-term solution for the condition or ailment being treated in the mammal.

Natural products continue to emerge as effective and preferred treatment options for all conditions, especially metabolic disease conditions, including weight loss and obesity, due to their potency, low toxicity, little to no side effects and ability to use them for long periods of time without dramatically lessening their effectiveness. Dyglomera (an extract of Dichrostachys glomerata), a popular Cameroonian spice, and CQR-300 (an extract of Cissus quadrangularis), an ornamental and medicinal plant that grows indigenously in Africa and Asia, have been shown to have tremendous positive effects on weight loss. Youvop et al. (2023) showed that Dyglomera (an extract of Dichrostachys glomerata) induced 22.85% weight loss in 60 subjects in 12 weeks. In a double-blind placebo-controlled study involving 35 subjects (Nash et al., 2019), CQR-300 (an extract of Cissus quadrangularis) reduced body fat by 12.8% in 8 weeks. The mechanism of these two extracts is not fully understood. It has been proposed that Dyglomera (an extract of Dichrostachys glomerata) and CQR-300 (an extract of Cissus quadrangularis) are anorectic. Additionally, CQR-300 (an extract of Cissus quadrangularis) was shown to boost serotonin levels (Kuate et al., 2015). Serotonin has received much attention in weight loss research in the past. It has been implicated for its appetite-suppressing effect on the arcuate nucleus hypothalamus, a region responsible for food intake and energy expenditure (Conde et al., 2023). Up to the present, no study has investigated the effect of Dyglomera (an extract of Dichrostachys glomerata) or CQR-300 (an extract of Cissus quadrangularis) on GLP-1 level or DPP-4 activity. Among other things, the results and data from the experiments described herein demonstrate the effects of Dyglomera (an extract of Dichrostachys glomerata) and CQR-300 (an extract of Cissus quadrangularis) separately and independently on blood GLP-1 levels, and their effect on the enzyme dipeptidyl phosphate-4 (DPP-4) in male Wistar rats. The results and data shown herein demonstrate that both Dyglomera (an extract of Dichrostachys glomerata) and CQR-300 (an extract of Cissus quadrangularis) each separately act as potent, safer alternatives for weight loss and obesity management.

Additionally, an aqueous extract of Dichrostachys glomerata (hereafter, “Dyglomera”) and an extract of Cissus quadrangularis (hereafter, “CQR-300”) each separately have been shown to provide a variety of health benefits to mammals, including lower blood glucose levels and induce weight loss. The data shown and disclosed herein demonstrate a significant loss of visceral fat, as measured by, among other things, a 10.23% reduction in waist circumference.

Additionally, data from the experiments shown and described herein show that both Dyglomera and CQR-300 each separately have various weight loss and health benefits that are comparable to Novo Nordisk's Rybelsus (semaglutide—C187H291N45O59), a glucagon-like peptide-1 (GLP-1) receptor agonist. Dyglomera and CQR-300, derived from natural plant extracts, provides a safer natural dietary supplement that does not cause the various side effects that pharmaceuticals, like Novo Nordisk's Rybelsus (semaglutide—C187H291N45O59) and other pharmaceutical drugs cause and can be used by mammals for longer periods of time without losing their effectiveness as compared to pharmaceuticals, all of which provides a more effective, safer and likely preferred alternative to pharmaceuticals like semaglutide as a GLP-1 receptor agonist.

### SUMMARY OF THE INVENTION

In one embodiment of the present disclosure, a composition for reducing visceral fat in a mammal is provided. The composition comprises an effective amount of Cissus quadrangularis, which is provided as an oral dosage unit in the form of a pill, capsule, liquid, lozenge or tablet.

In another embodiment of the present disclosure, a composition for reducing visceral fat in a mammal is provided. The composition comprises an effective amount of an extract of Cissus quadrangularis, which is provided as an oral dosage unit in the form of a pill, capsule, liquid, lozenge or tablet.

In an aspect of at least one embodiment of the present disclosure, the effective amount of the composition is about 0.7 to 8.0 mg/kg of body weight of the mammal taking the Cissus quadrangularis daily.

In an aspect of at least one embodiment of the present disclosure, the effective amount of the composition is about 200 mg to 600 mg of Cissus quadrangularis daily.

In another aspect of at least one embodiment of the present disclosure, the Cissus quadrangularis composition is an aqueous extract of Cissus quadrangularis.

In another aspect of at least one embodiment of the present disclosure, the Cissus quadrangularis composition is an ethanol extract of Cissus quadrangularis.

In another aspect of at least one embodiment of the present disclosure, the Cissus quadrangularis composition is comprised of Cissus quadrangularis fruit pods.

In another aspect of at least one embodiment of the present disclosure, the Cissus quadrangularis composition is comprised of dried Cissus quadrangularis fruit pods.

In another aspect of at least one embodiment of the present disclosure, the composition is provided to a mammal for at least 2 weeks.

In another embodiment of the present disclosure, a method for reducing visceral fat in a mammal is provided. The method comprises providing or prescribing an effective amount of an extract of Cissus quadrangularis fruit pods to a mammal in need thereof.

In an aspect of least one embodiment of the present disclosure, the composition is provided or prescribed as an oral dosage unit in the form of a pill, capsule, liquid, lozenge or tablet.

In another aspect of at least one embodiment of the present disclosure, the methods and compositions include providing or prescribing to a mammal, a composition containing about 2.6 mg of an extract of Cissus quadrangularis fruit pods, per kg of body weight of the mammal, daily.

In another aspect of at least one embodiment of the present disclosure, the method provides or prescribes a composition of about 200 mg to 600 mg of an extract of Cissus quadrangularis fruit pods to the mammal, daily.

In another aspect of at least one embodiment of the present disclosure, the method provides or prescribes a composition containing an ethanol extract of the Cissus quadrangularis fruit pods.

In another aspect of at least one embodiment of the present disclosure, the method provides or prescribes a composition of dried Cissus quadrangularis fruit pods.

In another aspect of at least one embodiment of the present disclosure, the method provides or prescribes a composition to a mammal for at least 2 weeks.

In another aspect of at least one embodiment of the present disclosure, the method provides or prescribes a composition to a mammal for at least 3 weeks.

In another aspect of at least one embodiment of the present disclosure, the method provides or prescribes a composition by a medical doctor or health care professional.

It should be appreciated that while specific embodiments have been discussed and disclosed herein, the scope of the present invention can only be defined by the claims of the present application and the scope of the inventions described herein are broader that the specific embodiments that have been discussed and disclosed herein.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a table presenting the effects of Dyglomera (an extract of Dichrostachys glomerata) and CQR-300 (an extract of Cissus quadrangularis), each separately, on food intake. *p<0.05: significantly different compared with the control group in the same column.

FIG. 2 is a table presenting the effects of Dyglomera (an extract of Dichrostachys glomerata) and CQR-300 (an extract of Cissus quadrangularis), each separately, on body weight. *p<0.05: significantly different compared with the control group in the same column.

FIG. 3 is a table presenting the effects of Dyglomera (an extract of Dichrostachys glomerata) and CQR-300 (an extract of Cissus quadrangularis), each separately, on visceral fat mass. *p<0.05: significantly different compared with the control group in the same column.

FIG. 4 is a table presenting the effects of Dyglomera (an extract of Dichrostachys glomerata) and CQR-300 (an extract of Cissus quadrangularis), each separately, on blood lipids. *p<0.05: significantly different compared with the control group in the same column; *p<0.05: significantly different compared with the CQR-300 (an extract of Cissus quadrangularis) in the same column.

FIG. 5 is a table presenting the effects of Dyglomera (an extract of Dichrostachys glomerata) and CQR-300 (an extract of Cissus quadrangularis), each separately, on fasting blood glucose. *p<0.05: significantly different compared with the control group in the same column.

FIG. 6 is a table presenting the effects of Dyglomera (an extract of Dichrostachys glomerata) and CQR-300 (an extract of Cissus quadrangularis), each separately, on GLP-1 level. *p<0.05: significantly different compared with the control group in the same column; #p<0.05: significantly different compared with the CQR-300 (an extract of Cissus quadrangularis) in the same column.

FIG. 7 is a table presenting the effects of Dyglomera (an extract of Dichrostachys glomerata) and CQR-300 (an extract of Cissus quadrangularis), each separately, on DPP-4 activity. *p<0.05: significantly different compared with the control group in the same column; #p<0.05: significantly different compared with the CQR-300 (an extract of Cissus quadrangularis) in the same column.

### DETAILED DESCRIPTION OF THE INVENTION

The following description is not to be taken in a limiting sense, but is made merely for the purpose of illustrating the general principles and variations of the inventions disclosed herein. Various inventive features are described below that can each be used independently of one another or in combination with other features or aspect of the inventions and various embodiments disclosed herein.

### Experimental Methods

The plant material used in the studies and associated data shown and described herein consisted of Dichrostachys glomerata or Cissus quadrangularis. The dried fruit pods of Dichrostachys glomerata and Cissus quadrangularis were bought at the market of Mfoundi (locality of Yaoundé, capital of Cameroon) in September 2022. The fruits were ground into a uniform powder to increase the surface area of the sample with extraction solvents. The solvents used were distilled water and 95% ethanol. Plant dried material (100 g) was weighed and poured into 2 sterile glass beakers, each containing 400 ml of 95% ethanol and water, respectively, for the preparation of ethanolic and aqueous extracts. Each solution was shaken vigorously for 24 h and 48 h, respectively, for aqueous and ethanol extracts, and filtered with Whatmann filter paper No.3. The filtrates obtained were evaporated using a rotary evaporator (BUCHI Rotavapor R-114, Switzerland) at 60° C. until the extracts became completely dry.

Twenty-four male Wistar albino rats of 190-215 g body weight were obtained from the central animal house of the BPA Department of the Faculty of Science, UY1. The animals were handled according to the European Union Animal Care (CEE Council 86/609) guideline adopted by the Cameroon National Ethics Committee. Animals were housed under standard conditions with relative humidity (50 %) at 25° C. temperature and acclimatized to 12 h light/dark cycle 10 days prior to the day of experimentation. They were given access to food (standard rat chow pellets) and water ad libitum.

The experiments and associated data obtained from those experiments disclosed herein, was carried out over a period of 6 weeks, and was approved by the Institutional Animal Ethical Committee. A total of twenty-four (24) male Wistar albino rats of 190-215 g body weight were used. The animals were divided into the following groups: (1) Group 1 or control group: eight (08) rats+normal diet; (2) Group 2 or test group 1 [Dyglomera (an extract of Dichrostachys glomerata)]: eight (08) rats+normal diet+6 mg/kg of PC of Dyglomera (an extract of Dichrostachys glomerata); Group 3 or test group 2 [CQR-300 (an extract of Cissus quadrangularis)]: eight (08) rats+normal diet+5 mg/kg of PC of CQR-300 (an extract of Cissus quadrangularis).

After randomly dividing the rats according to their average weight, three (03) rats in each group were sacrificed by cervical dislocation for assessment of baseline (week 0) visceral fat. The rest of the experiment was carried out with five (5) rats in each group. The rat body weights were measured at baseline (week 0) and at weeks 1, 2, 3, 4 and at the end (week 6). The food intake was measured daily and summed to obtain weekly totals. The fasting blood glucose was assessed using (week 0, 2, and 6). At the end of the experiment, the rats were sacrificed by cervical dislocation. The end visceral fat (week 6) was assessed, and the blood was collected in dry tubes for serum preparation. The serum metabolic parameters assessed were total cholesterol, triglycerides, HDL-c, LDL-c, GLP-1 levels and DPP-4 activity.

The effect on visceral fat was assessed by collecting and weighing the visceral fat after the animals had been sacrificed.

The fasting blood glucose level of rats was assessed and estimated on week 0, 2, and 6 by glucose oxidase-peroxidase enzymatic method using one touch glucometer. The effect on fasting blood lipids (cholesterol, triglycerides, HDL-c, and LDL-c,) was assessed using Chronolab kits.

The effect of the extracts on GLP-1 level, and DPP-4 activity was also assessed at the baseline (week 0), week 2, week 4, and week 6. The blood samples were obtained from tail vein after the administration of the extracts (Bhat et al., 2018). The GLP-1 level assessment was determined by Ray Bio Rat GLP-1 ELISA kit.

The DPP-4 activity was carried out using Cayman's DPP-4 inhibitor screening assay kit based on the manufacturer's instructions. Briefly, the serums (10 μL) obtained from blood samples (test 1, 2 and control groups), and the standard inhibitor (10 μL) were introduced in 1 mL of the assay buffer. The samples, positive control and the standard inhibitor (given in the kit) were incubated with DPP-4 enzyme for 20 minutes at room temperature. The fluorogenic substrate provided with the kit was added, and the plate was read for 30 minutes using a fluorometer (Biotek Synergy H1 microplate reader) at Ex:380/EM:460 nm. The percentage of remaining activity was calculated using the following formula: % activity remaining=(slope of test sample/positive control slope)×100. NB: (In the positive control, the sample was replaced by the buffer).

The SPSS software version 20.0 for Windows was used for statistical analysis. The ANOVA was used for the descriptive analysis and the comparison between the control, Dyglomera (an extract of Dichrostachys glomerata), and CQR-300 (an extract of Cissus quadrangularis), groups was performed by the post hoc LSD test. The results were expressed as a mean±standard error on mean. The statistical significance was considered at p<0.05. The graph was plotted and the data processed using Excel.

### Experimental Results

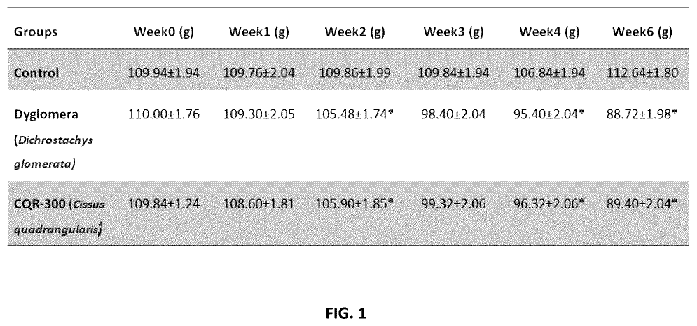

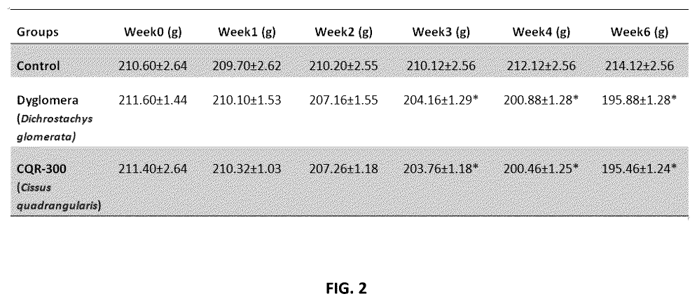

FIG. 1 table and FIG. 2 table present the results of the effects of the Dyglomera (an extract of Dichrostachys glomerata), and CQR-300 (an extract of Cissus quadrangularis) on food intake and body weight respectively. After 6 weeks of intake, the decrease in food intake in the Dyglomera (an extract of Dichrostachys glomerata), and CQR-300 (an extract of Cissus quadrangularis) compared to the control groups was significant. However, the decrease of rat's body weight was similar in the Dyglomera (an extract of Dichrostachys glomerata), and CQR-300 groups (an extract of Cissus quadrangularis).

After 6 weeks of intake, a decrease in body weight in the Dyglomera (an extract of Dichrostachys glomerata), and CQR-300 (an extract of Cissus quadrangularis) compared to the control groups was significant. However, FIG. 2 shows a decrease of rat's body weight was similar in the Dyglomera (an extract of Dichrostachys glomerata), and CQR-300 (an extract of Cissus quadrangularis) groups.

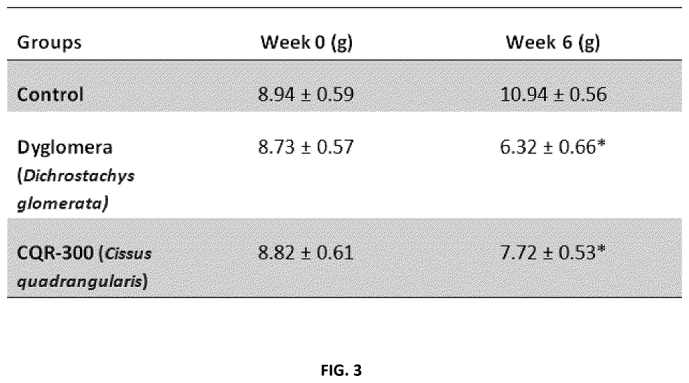

FIG. 3 table presents the results of the effects of the Dyglomera (an extract of Dichrostachys glomerata), and CQR-300 (an extract of Cissus quadrangularis) on visceral fat mass. After 6 weeks of intake, the decrease in visceral fat mass in the Dyglomera (an extract of Dichrostachys glomerata), and CQR-300 (an extract of Cissus quadrangularis) compared to the control groups was significant.

While overall weight reduction is often prescribed as a panacea, recent research has increasingly highlighted the specific dangers of visceral fat. This deep abdominal fat, encasing vital organs, is metabolically distinct from subcutaneous fat and poses unique health threats. Research has shown that targeting visceral fat specific is difficult to do and provides significant health benefits and advantages over reducing fat or weight generally.

Fat, while often viewed as a homogenous tissue, is comprised of different depots with varying metabolic functions. Subcutaneous fat, located beneath the skin, primarily serves as energy storage. Conversely, visceral fat, nestled within the abdominal cavity, is intimately involved in metabolic processes and having too much visceral fat negatively effects a person's health and metabolism.

Visceral adipose tissue (VAT) is highly metabolically active, secreting a plethora of adipokines, including inflammatory cytokines, leptin, and adiponectin. These hormones influence insulin resistance, inflammation, and appetite regulation. Subcutaneous adipose tissue (SAT) is relatively metabolically quiescent. Visceral adipose tissue is distributed around vital organs such as the liver, pancreas, and intestines, while SAT is primarily located beneath the skin.

Excessive visceral adipose tissue is strongly linked to metabolic syndrome, type 2 diabetes, cardiovascular disease, and non-alcoholic fatty liver disease. While SAT can contribute to these conditions, its impact is generally less pronounced. The health risks associated with visceral fat are multifaceted and stem from its endocrine and inflammatory properties.

Additionally, visceral adipose tissue contributes to insulin resistance by releasing inflammatory cytokines that impair insulin signaling in peripheral tissues. This leads to hyperinsulinemia, a precursor to type 2 diabetes.

Visceral adipose tissue or fat is also associated with increased production of proinflammatory adipokines, which promote atherosclerosis, a condition characterized by plaque buildup in arteries. Moreover, visceral adipose tissue or fat is linked to hypertension and dyslipidemia, key risk factors for cardiovascular disease.

Excessive visceral adipose tissue can also lead to non-alcoholic fatty liver disease (NAFLD), a condition characterized by fat accumulation in the liver. This can progress to non-alcoholic steatohepatitis (NASH), a more severe form of liver inflammation. Visceral adipose tissue is a chronic low-grade inflammation site, contributing to systemic inflammation. This inflammatory state underlies the development of various chronic diseases.

Visceral fat is a metabolically active tissue that poses significant health risks. Targeting this specific fat depot, rather than solely focusing on overall weight loss, is essential for preventing and managing chronic diseases. By understanding the distinct characteristics and metabolic consequences of visceral fat, healthcare providers and individuals can implement effective strategies to reduce this harmful fat and improve overall health in a mammal. Accordingly, while overall weight loss can be beneficial, there exists a need to reduce visceral fat specifically to improve the health of mammals.

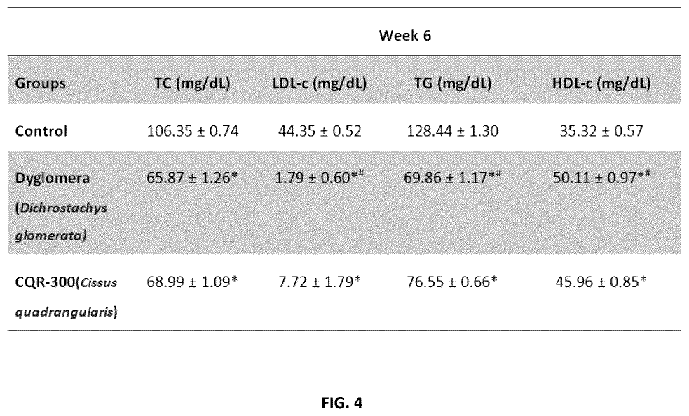

FIG. 4 table presents the results of the effects of the Dyglomera (an extract of Dichrostachys glomerata), and CQR-300 (an extract of Cissus quadrangularis) on lipid metabolic parameters. After 6 weeks of intake, the decrease in blood lipids (TC, LDL-c, and TG), and increase of HLD-c in the Dyglomera (an extract of Dichrostachys glomerata), and CQR-300 (an extract of Cissus quadrangularis) compared to the control group were significant (table 4). These changes in TC, LDL-c, TG and HDL-c were significant when compared Dyglomera (an extract of Dichrostachys glomerata), and CQR-300 (an extract of Cissus quadrangularis) to the control group.

Cardiovascular disease remains a leading cause of mortality globally. While the role of various risk factors is well-established, the positive or good lipid profile, a snapshot of blood cholesterol and triglycerides, has been shown to be effective at predicting and preventing heart disease. There are many reasons why weight loss alone may not suffice in optimizing a mammal's lipid profile values.

Blood lipids, essential for various bodily functions, are transported in lipoproteins. The key components of a lipid profile include total cholesterol (hereafter, “TC”): A combined measure of all cholesterol types. Low-density lipoprotein cholesterol (LDL-c) is often referred to as “bad cholesterol,” as it contributes to plaque buildup in arteries. High-density lipoprotein cholesterol (HDL-c) is often referred to as “good cholesterol,” as it helps remove cholesterol from the arteries. Triglycerides (hereafter, “TG”) is a type of fat stored in fat cells.

A lipid profile that favors lower TC, LDL-c, and TG levels, coupled with higher HDL-c, is associated with a significantly reduced risk of cardiovascular disease, irrespective of a mammal's actual weight.

Additionally, LDL-c plays a pivotal role in atherosclerosis, a process where plaque builds up in artery walls. Lower LDL-c levels directly inhibit this process, reducing the risk of heart attacks and strokes. HDL-c is essential for reverse cholesterol transport, removing excess cholesterol from the arteries and transporting it to the liver for elimination. Higher HDL-c levels enhance blood flow and protect against heart disease.

Chronic inflammation is linked to cardiovascular disease. Certain lipid profiles, particularly those with elevated triglycerides, can contribute to inflammation. Lowering TG levels can help mitigate this risk.

While weight loss is often associated with improvements in lipid profiles, the correlation is not straightforward. Weight loss can occur through various mechanisms, including loss of fat, muscle, or water. The impact on lipid profiles depends on the composition of weight loss. Weight loss can induce metabolic adaptations, such as increased hepatic lipid production or altered lipoprotein metabolism. These changes can counteract the positive effects of weight loss on lipids. Accordingly, there exist a need to reduce blood lipids (and possibly increase HDL levels) in a mammal to reduce the risk of cardiovascular disease and various related conditions, like inflammation.

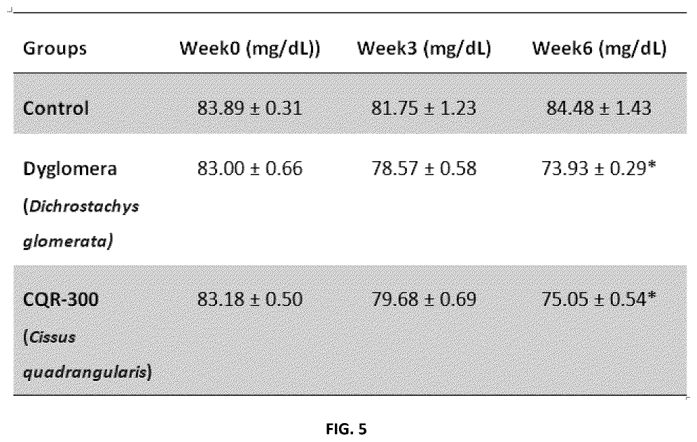

FIG. 5 table presents the results of the effects of Dyglomera (an extract of Dichrostachys glomerata), and CQR-300 (an extract of Cissus quadrangularis) on fasting blood glucose. After 6 weeks of intake, the decrease in fasting blood glucose in the Dyglomera (an extract of Dichrostachys glomerata), and CQR-300 (an extract of Cissus quadrangularis) compared to control group was significant.

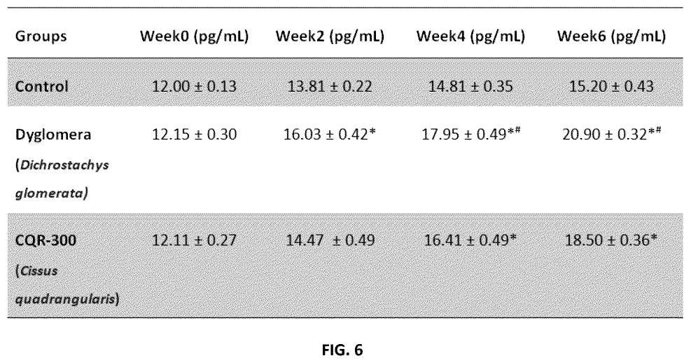

FIG. 6 presents the results of the effects of Dyglomera (an extract of Dichrostachys glomerata), and CQR-300 (an extract of Cissus quadrangularis) on GLP-1 level. After 6 weeks of intake, the increase of GLP-1 in the Dyglomera (an extract of Dichrostachys glomerata), and CQR-300 (an extract of Cissus quadrangularis) compared to control groups was significant. These results showed a significant increase in GLP-1 from week 2 to week 6 in treated groups.

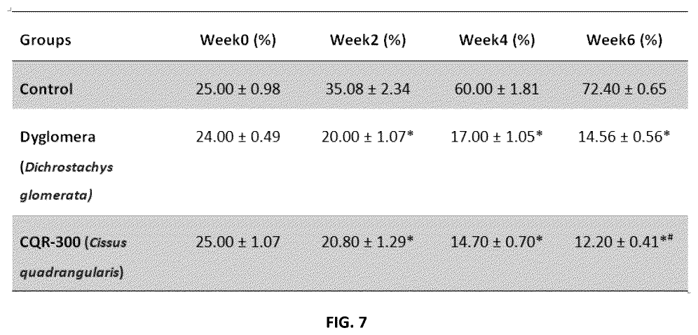

FIG. 7 presents the results of the effects of Dyglomera (an extract of Dichrostachys glomerata), and CQR-300 (an extract of Cissus quadrangularis) on DPP-4 activity These experiments and the associated data obtained from the experiments, which are described and disclosed herein, evaluated the effects of Dyglomera (an extract of Dichrostachys glomerata) separately, and CQR-300 (an extract of Cissus quadrangularis) separately on blood GLP-1 levels, and each of their effects on the enzyme dipeptidyl phosphate-4 (DPP-4) in male Wistar rats as potent alternatives in the management of obesity. The findings showed a comparable effect of CQR-300, and Dyglomera (an extract of Dichrostachys glomerata) on food intake and body weight. In comparison to numerous studies, our findings maintain consistency in the efficacy of these treatments in reducing appetite and inducing weight loss.

The Dyglomera (an extract of Dichrostachys glomerata) and CQR-300 (an extract of Cissus quadrangularis) have been reported for their weight loss in several studies. Youovop et al. (2023) showed a 22.85% weight loss in 60 subjects after 12 weeks of administering Dyglomera (an extract of Dichrostachys glomerata). In Nash et al. (2019), CQR-300 reduced body fat by 12.8% in a double-blind placebo-controlled study involving 35 subjects after 8 weeks. Kim et al. (2022) proposed that Dyglomera (an extract of Dichrostachys glomerata) reduced food intake due to the secretion of adiponectin, which stimulates the phosphorylation of AMPK. AMPK has been found to improve insulin sensitivity and induce the catabolism of fat for energy expenditure. Furthermore, Kola (2008) explained that AMPK is highly expressed in the regions of the brain responsible for food intake, serving as a glucose nutrient sensor and regulating appetite. A similar pathway is used to justify the anorexic effects of CQR-300 in Lee et al. (2018). Additionally, Kuate et al. (2015) reported an increase in serotonin levels with the continued administration of CQR-300 (an extract of Cissus quadrangularis) compared to the placebo group. Serotonin has received much attention in weight loss research in the past, as it has been implicated in the suppression of appetite and reduced food intake through its action on the arcuate nucleus hypothalamus, a region responsible for food intake and energy expenditure (Conde et al., 2023).

Regarding visceral fat mass, Dyglomera (an extract of Dichrostachys glomerata) showed better performance in the reduction of visceral fat compared to the other treatment groups. The mechanism of this effect may be associated with the lipolytic effects of adiponectin activity and the upregulation of AMPK as mentioned earlier.

Hyperglycemia, insulin resistance, and dyslipidemia are among the major complications associated with obesity. This study shows a significant improvement in blood lipid metabolism in Dyglomera (an extract of Dichrostachys glomerata) and CQR-300 (an extract of Cissus quadrangularis), but not in the control groups. The Dyglomera (an extract of Dichrostachys glomerata) and CQR-300 (an extract of Cissus quadrangularis) are better antihyperlipidemic agents, in agreement with earlier studies (Azantsa et al., 2015, Kuate et al., 2015; Youvop et al., 2023; Lee et al., 2018). The aforementioned studies agreed that Dyglomera (an extract of Dichrostachys glomerata) and CQR-300 (an extract of Cissus quadrangularis) activated the down regulation of adipogenesis-associated proteins such as PPAR gamma, C/EBP alpha, and SREBP-1c through the action of phosphorylated AMPK.

All treatment groups, except the control, showed significant improvement in glucose uptake as indicated by lower blood glucose at week 6. In Kim et al. (2022), Dyglomera (an extract of Dichrostachys glomerata) reduced blood glucose by 27.67% in obese patients in a 12-week study through the action of adiponectin. As mentioned earlier, adiponectin is an abundant hormone secreted in fat cells. Adiponectin is well known for improving insulin sensitivity by suppressing gluconeogenesis in the liver, stimulation of AMPK, and fatty acid oxidation in skeletal muscles (Ruan and Dong, 2016). In Oben et al. (2007), CQR-300 (an extract of Cissus quadrangularis) reduced fasting blood glucose in obese subjects by 14.6% after 6 weeks. Similar to Dyglomera (an extract of Dichrostachys glomerata), Lee et al. (2016) explained that CQR-300 (an extract of Cissus quadrangularis) regulated the secretion of adiponectin and leptin, which play key roles in improving insulin sensitivity and enhancing glucose uptake.

The GLP-1 receptor agonists (GLP-1RAs) have become popular in the clinical management of obesity (van Bloemendaal et al., 2014). In this study, there was a steady increase in the GLP-1 level across Dyglomera (an extract of Dichrostachys glomerata), and CQR-300 (an extract of Cissus quadrangularis) compared to the control groups. The underlying mechanism for the increased GLP-1 level appears to be an inhibitory activity of the enzyme DPP-4, as shown by the results obtained in this study

The results from the study and experiments performed demonstrated that effects of both Dyglomera (an extract of Dichrostachys glomerata), and CQR-300 in reducing physiologic (food intake, body weight, visceral fat), and metabolic (fasting blood glucose, blood lipids) parameters resulted from their potential to increase GLP-1 secretion and inhibition of DPP-4 activity. This increase in the level of GLP-1 secretion was most effective with Dyglomera (an extract of Dichrostachys glomerata), while CQR-300 (an extract of Cissus quadrangularis) showed the best inhibition of DPP-4.

While overall weight loss is often a goal for many, it's important to understand that reducing weight is not synonymous with reducing visceral fat. Visceral fat, a type of fat that accumulates deep within the abdominal cavity, poses a significant health risk even in individuals with a normal body mass index (BMI). Accordingly, a reduction in weight does not mean that there will be a reduction in visceral fat, which is known to cause significant health problems.

Visceral fat, often referred to as “deep belly fat,” is distinct from subcutaneous fat, the more visible kind found just beneath the skin. It surrounds vital organs such as the liver, pancreas, and intestines. Unlike subcutaneous fat, which primarily serves as insulation and energy storage, visceral fat is metabolically active and can produce inflammatory substances that contribute to various health problems.

While overall weight loss can sometimes lead to a reduction in visceral fat, the two are not always directly correlated. It's possible for individuals to lose weight, particularly through muscle mass loss, while maintaining or even increasing their visceral fat. This is because visceral fat is more resistant to weight loss efforts compared to subcutaneous fat.

Excess visceral fat is linked to a multitude of health problems, including but not limited to: Metabolic Syndrome: (1) A cluster of conditions that increase the risk of heart disease, stroke, and type 2 diabetes. These conditions often include high blood pressure, high blood sugar, excess body fat around the waist, and abnormal cholesterol levels, (2) Cardiovascular Disease: Visceral fat is associated with increased risk of heart attack, stroke, and peripheral artery disease. It can contribute to atherothrombosis, the buildup of plaque in the arteries, which can lead to blockages and reduced blood flow, (3) Type 2 Diabetes: The inflammatory substances produced by visceral fat can interfere with insulin sensitivity, making it more difficult for the body to regulate blood sugar levels. This can increase the risk of developing type 2 diabetes. (4) Liver Disease: Excessive visceral fat can lead to non-alcoholic fatty liver disease (NAFLD), a condition characterized by the accumulation of fat in the liver. NAFLD can progress to more serious conditions like cirrhosis and liver cancer, and (5) Certain Cancers: Some studies have linked excess visceral fat to an increased risk of certain cancers, including breast, colon, and pancreatic cancer.

While overall weight loss is a valuable health goal, it's essential to recognize that weight loss in general doesn't equal or guarantee a reduction in visceral fat. Excess visceral fat poses significant health risks, including cardiovascular disease, type 2 diabetes, and certain cancers. By adopting a healthy lifestyle working to reduce visceral fat in particular, the overall health of a mammal can be improved using the compositions and methods disclosed herein.

### References

Ahrén B., and Schmitz O. (2004). GLP-1 Receptor Agonists and DPP-4 Inhibitors in the Treatment of Type 2 Diabetes. Horm Metab Res, 36(11/12): 867-876. DOI:10.1055/s-2004-826178
Azantsa, B., Kuate, D., Chakokam, R., Paka, G., Bartholomew, B., & Nash, R. (2015). The effect of extracts of Irvingia gabonensis (IGOB131) and Dichrostachys glomerata (Dyglomera) on body weight and lipid parameters of healthy overweight participants. Functional Foods in Health and Disease, 5(6), 200-208.
Bhat G., Khan H., Alhomida A., Sharma P., Singh R., and Paray B. (2018). GLP-I secretion in healthy and diabetic Wistar rats in response to aqueous extract of Momordica charantia. BMC Complementary and Alternative Medicine, 18 (162), 1-8
Conde, K., Fang, S., & Xu, Y. (2023). Unraveling the serotonin saga: from discovery to weight regulation and beyond-a comprehensive scientific review. Cell & Bioscience, 13(1), 143.
Filipova, E., Uzunova, K., & Vekov, T. (2015). Comparative analysis of therapeutic efficiency and costs (experience in Bulgaria) of oral antidiabetic therapies based on glitazones and gliptins. Diabetology & Metabolic Syndrome, 7, 63.
Filippatos, T., Panagiotopoulou, T., & Elisaf, M. (2014). Adverse Effects of GLP-1 Receptor Agonists. The Review of Diabetic Studies, 11, 3-4.
Kim, H. L., Lee, S. K., Min, D. E., Choi, B. K., & Lee, D. R. (2022). Anti-Obesity Effect of Dyglomera Is Associated with Activation of the AMPK Signaling Pathway in 3T3-L1 Adipocytes and Mice with High-Fat Diet-Induced Obesity. Molecules, 27(10), 3288.
Kola, B. (2008). Role of AMP-activated protein kinase in the control of appetite. Journal of neuroendocrinology, 20(7), 942-951.
Kuate, D., Nash, R. J., Bartholomew, B., & Penkova, Y. (2015). The use of Cissus quadrangularis (CQR-300) in the management of components of metabolic syndrome in overweight and obese participants. Natural Product Communications, 10(7), 1934578X1501000737.
Lee, H. J., Le, B., Lee, D. R., Choi, B. K., & Yang, S. H. (2018). Cissus quadrangularis extract (CQR-300) inhibits lipid accumulation by downregulating adipogenesis and lipogenesis in 3T3-L1 cells. Toxicology Reports, 5, 608-614.
Lee, H. J., Lee, D. R., Choi, B. K., Park, S. B., Jin, Y. Y., Yang, S. H., & Suh, J. W. (2016). Cissus quadrangularis extracts decreases body fat through regulation of fatty acid synthesis in high-fat diet-induced obese mice. Journal of Applied Biological Chemistry, 59(1), 49-56.
Nash, R., Azantsa, B., Kuate, D., Singh, H., & Oben, J. (2019). The use of a stem and leaf aqueous extract of Cissus quadrangularis (CQR-300) to reduce body fat and other components of metabolic syndrome in overweight participants. The Journal of Alternative and Complementary Medicine, 25(1), 98-106.
Oben, J. E., Enyegue, D. M., Fomekong, G. I., Soukontoua, Y. B., & Agbor, G. A. (2007). The effect of Cissus quadrangularis (CQR-300) and a Cissus formulation (CORE) on obesity and obesity-induced oxidative stress. Lipids in Health and Disease, 6, 1-8.
Ruan, H., & Dong, L. Q. (2016). Adiponectin signaling and function in insulin target tissues. Journal of molecular cell biology, 8(2), 101-109.
Tkáč, I., & Raz, I. (2017). Combined Analysis of Three Large Interventional Trials With Gliptins Indicates Increased Incidence of Acute Pancreatitis in Patients With Type 2 Diabetes, Diabetes Care, 40, 284-286. DOI:10.2337/dc15-1707
Valerio, C., de Almeida, J., Moreira, R., Aguiar, L., Siciliano, L., Carvalho, D., & Godoy-Matos, A. (2017). Dipeptidyl peptidase-4 levels are increased and partially related to body fat distribution in patients with familial partial lipodystrophy type 2. Diabetol Metab Syndr, 9, 26. DOI 10.1186/s13098-017-0226-0
van Bloemendaal, L., Ten Kulve, J. S., La Fleur, S. E., Ijzerman, R. G., & Diamant, M. (2014). Effects of glucagon-like peptide 1 on appetite and body weight: focus on the CNS. J Endocrinol, 221(1), T1-16.
WHO (2021). Obesity and Overweight. https://www.who.int/news-room/fact-sheets/detail/obesity-and-overweight Accessed 21 Feb. 2024.
Yadav, D., & Lowenfels, A. (2013). The Epidemiology of Pancreatitis and Pancreatic Cancer. Gastroenterology, 144(6), 1252-1261. doi:10.1053/j.gastro.2013.01.068.
Youovop, J., Takuissu, G., Mbopda, C., Nwang, F., Ntentié, R., Mbong, M., Azantsa, B., Singh, H. & Oben, J (2023) The effects of Dyglomera (Dichrostachys glomerata extract) on body fat percentage and body weight: a randomized, double-blind, placebo-controlled clinical trial. Functional Foods in Health and Disease 13(6):334-346.

What is claimed is:

1. A composition for reducing visceral fat in a mammal, the composition comprising:
an effective amount of Cissus quadrangularis;
wherein the composition is provided as an oral dosage unit in the form of a pill, capsule, liquid, lozenge or tablet.

2. The composition of claim 1, wherein the effective amount of the composition is about 200 mg to 600 mg of Cissus quadrangularis daily.

3. The composition of claim 1, wherein the effective amount of the composition is about 2.6 mg of an extract of Cissus quadrangularis fruit pods, per kg of body weight of the mammal.

4. The composition of claim 1, wherein the Cissus quadrangularis composition is an aqueous ethanol extract of Cissus quadrangularis.

5. The composition of claim 1, wherein the Cissus quadrangularis composition is comprised of Cissus quadrangularis fruit pods.

6. The composition of claim 1, wherein the Cissus quadrangularis composition is comprised of dried Cissus quadrangularis fruit pods.

7. The composition of claim 1, wherein the composition is provided to a mammal for at least 2 weeks.

8. A composition for reducing visceral fat in a mammal, the composition comprising:
an effective amount of an extract of Cissus quadrangularis;
wherein the composition is provided as an oral dosage unit in the form of a pill, capsule, liquid, lozenge or tablet.

9. The composition of claim 8, wherein the effective amount of the composition is about 2.6 mg of an extract of Cissus quadrangularis fruit pods, per kg of body weight of the mammal.

10. The composition of claim 8, wherein the Cissus quadrangularis composition is an ethanol extract of Cissus quadrangularis.

11. The composition of claim 8, wherein the extract of Cissus quadrangularis composition is comprised of an extract of Cissus quadrangularis fruit pods.

12. The composition of claim 8, wherein the extract of Cissus quadrangularis composition is comprised of an extract of dried Cissus quadrangularis fruit pods.

13. The composition of claim 8, wherein the composition is provided to a mammal for at least 2 weeks.

14. A method for reducing visceral fat in a mammal, the method comprising:
providing or prescribing an effective amount of an extract of Cissus quadrangularis fruit pods;
wherein the composition is provided or prescribed as an oral dosage unit in the form of a pill, capsule, liquid, lozenge or tablet.

15. The method of claim 14, wherein the effective amount of the composition is about 2.6 mg of an extract of Cissus quadrangularis fruit pods, per kg of body weight of the mammal.

16. The method of claim 14, wherein the extract of Cissus quadrangularis fruit pods composition is an ethanol extract of the Cissus quadrangularis fruit pods.

17. The method of claim 14, wherein the extract of Cissus quadrangularis fruit pods is obtained from dried Cissus quadrangularis fruit pods.

18. The method of claim 14, wherein the composition is provided or prescribed to a mammal for at least 2 weeks.

19. The method of claim 14, wherein the composition is provided or prescribed to a mammal for at least 3 weeks.

20. The method of claim 14, wherein the composition is provided or prescribed by a medical doctor or health care professional.
